(19) United States
(12) Patent Application Publication
(10) Pub. No.: US 2014/0018726 A1
(43) Pub. Date: Jan. 16, 2014

(54) DEVICE FOR THE TRANSPORT OF SMALL QUANTITIES OR INDIVIDUAL QUANTITIES OF MEDICAL GAS

(71) Applicant: Timothy J. Warren, Easton, PA (US)

(72) Inventor: Timothy J. Warren, Easton, PA (US)

(73) Assignee: AIR LIQUIDE HEALTHCARE AMERICA CORPORATION, Houston, TX (US)

(21) Appl. No.: 14/001,151

(22) PCT Filed: Feb. 23, 2012
(86) PCT No.: PCT/US12/26258

§ 371 Date: Oct. 3, 2013

Related U.S. Application Data
(60) Provisional application No. 61/445,842, filed on Feb. 23, 2011.

(51) Int. Cl. A61F 9/007; F17C 3/00

(52) U.S. Cl. CPC A61F 9/00727; F17C 3/00
USPC 604/26; 206/6; 53/408

(57) ABSTRACT

The present invention relates to a gas composition device that can be used for the sterile transport and storage of a gas composition that will be used with regard to a human or an animal. The device of the present invention consist essentially of a gas container (2) positioned within an cavity of an outer package, the gas container containing a gas composition within an internal reservoir of the gas container and being surrounded in the cavity of the outer package by the same gas composition.

### CROSS REFERENCE TO RELATED APPLICATIONS

This application is a 371 of International PCT Application PCT/US2012/026258, filed Feb. 23, 2012, which claims priority to U.S. provisional application No. 61/445,842, filed Feb. 23, 2011, the entire contents of which are incorporated herein by reference

### FIELD OF THE INVENTION

The present invention relates to a gas composition device for the sterile transport and storage of a gas composition to be used with regard to a human or an animal.

### SUMMARY

The present invention relates to a gas composition device that can be used for the sterile transport and storage of a gas composition that will be used with regard to a human or an animal. The device of the present invention consist essentially of a gas container positioned within an cavity of an outer package, the gas container containing a gas composition within an internal reservoir of the gas container and being surrounded in the cavity of the outer package by the same gas composition. The reasoning behind including the gas composition as the atmosphere for storage of the gas container within the cavity of the outer package is to assure the integrity of the gas composition within the gas container. More specifically, the likelihood of contamination of the gas composition in the gas container occurring is decreased dramatically as the material which could diffuse into the internal reservoir due to a breech of any seal in the gas container over a period of time when the device is stored (the intended shelf life) is the same in content as the actual gas composition contained in the internal reservoir. Therefore, even if there is a breach of any seal in the gas container, the integrity of the gas composition is not compromised. The integrity of the gas composition is further supported by the utilization of a material to construct the outer package that is sufficiently or completely impermeable to contaminants.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 illustrates one embodiment of the device 1 of the present invention includes a gas container 2 having an internal reservoir 3 for the gas composition 4 and a means for allowing the withdrawal of the gas composition 5, an outer package 6 that includes an cavity 7 for housing the gas container 2 and for housing an additional amount of the gas composition 4 (depicted by shading in the cavity 7);

FIG. 2 illustrates the means for allowing the withdrawal of the gas composition 5 will likely be located at either the first end 8 or the second end 9 of the cylindrical gas container 2 (shown with regard to the first end 8);

FIG. 3 illustrates an alternative embodiment of the present gas container 2 in which the gas container 2 is round or oval (not having a clear first end or a clear second end) with an internal reservoir 3 disposed therein and a means for allowing the withdrawal of the gas composition 5 being located at any position on the gas container 2;

FIG. 4a illustrates the means for allowing the withdrawal of the gas composition 5 from the gas container 2, the means 5 being a valve 10;

FIG. 4b illustrates the means for allowing the withdrawal of the gas composition 5 from the gas container 2, the means 5 being a barrier that is capable of being punctured 11 to allow for the withdrawal of the gas composition 4; and

FIG. 5 illustrates an alternative embodiment of the present gas container 2.

### DETAILED DESCRIPTION OF THE INVENTION

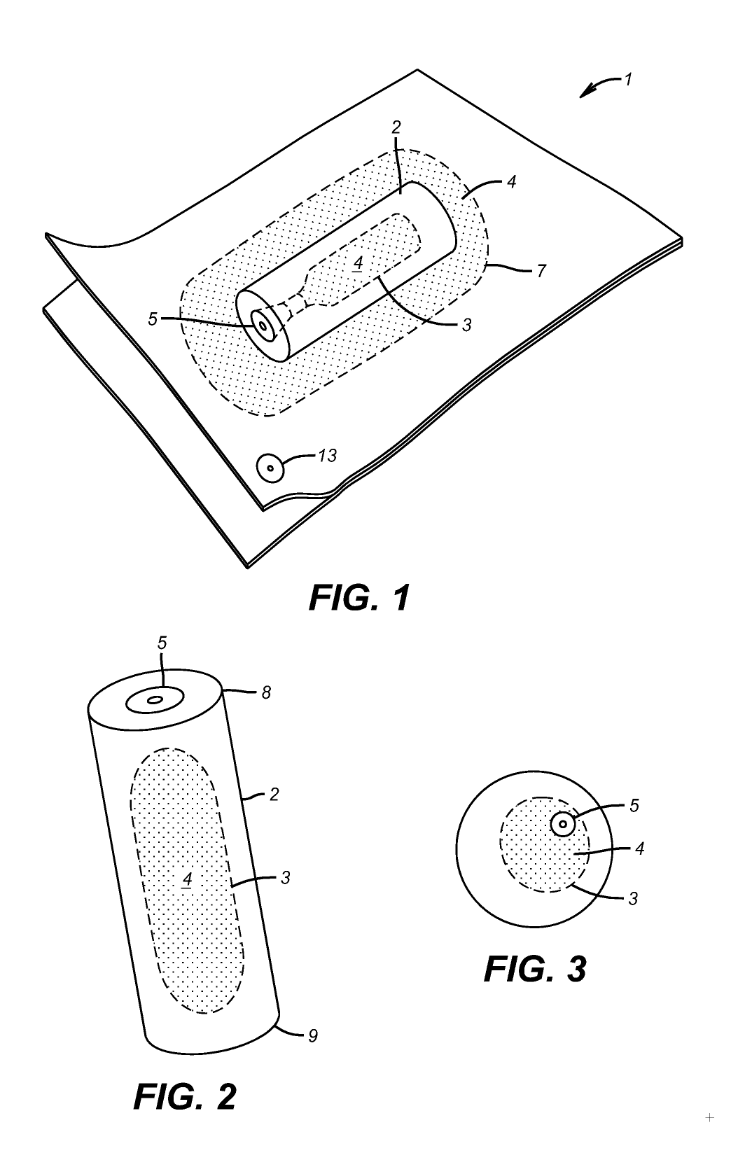

For purposes of the present invention, reference will be made to the figures contained herein when discussing the present invention and methods for using the same. Note however, that the present invention is not meant to be limited by the figures contained herein and that reference to such figures is merely for illustrative purposes. With reference to FIG. 1, one embodiment of the device 1 of the present invention includes a gas container 2 having an internal reservoir 3 for the gas composition 4 and a means for allowing the withdrawal of the gas composition 5, an outer package 6 that includes an cavity 7 for housing the gas container 2 and for housing an additional amount of the gas composition 4 (depicted by shading in the cavity 7). With regard to this embodiment, the gas container 2 utilized is any gas container 2 that has an internal reservoir 3 disposed therein for holding the gas composition 4 and also includes a means for allowing the withdrawal of the gas composition 5. While the present invention is not meant to be limited in any way by the shape of the gas container 2, in one embodiment of the present invention, the gas container 2 utilized and depicted in FIG. 2 is a cylindrical gas container 2 that has a first end 8, a second end 9 and an internal reservoir 3 disposed between the first end 8 and the second end 9 of the cylindrical gas container 2. In addition, in this embodiment, the means for allowing the withdrawal of the gas composition 5 will likely be located at either the first end 8 or the second end 9 of the cylindrical gas container 2 (shown with regard to the first end 8 in FIG. 2). In an alternative embodiment of the present gas container 2 as shown in FIG. 3, the gas container 2 may be round or oval (not having a clear first end or a clear second end) with an internal reservoir 3 disposed therein and a means for allowing the withdrawal of the gas composition 5 being located at any position on the gas container 2. In the preferred embodiment, the gas container 2 will be rectangular or cylindrical in shape.

As the intent of the present invention is to provide a small amount of gas composition 4 for use, the dimensions of the corresponding gas container 2 will for all practical purposes be small or limited in scope (having sufficient dimensions to allow for the containment of the amount of gas composition 4 needed for the particular method of use noted). More specifically, when the gas container 2 is a round gas container 2 as shown in FIG. 3, the width of the gas container 2 will likely be from about ½ inch to about 2½ inches, preferably from 1 inch to about 2 inches. In the embodiment where the gas container 2 is a rectangular or cylindrical gas container 2 as shown in FIG. 2, the length of the gas container 2 will likely range from about 2 inches to about 12 inches, preferably from about 2 inches to about 4 inches and the width of the cylindrical gas container 2 will likely be from about ½ inches to about 2½ inches, preferably from about 1 inch to about 2 inches. Note that the shape of the internal reservoir 3 of the gas container 2 is not critical to the present invention. Typically, any shape is allowed as long as this shape is such that it is fully contained within the gas container 2. With regard to the gas container 2, this gas container 2 may be fabricated of any material which is impermeable or substantially impermeable to the gas composition 4. Accordingly, the gas container 4 can be made of a metal such as stainless steel, a strong plastic, glass or other suitable materials capable of maintaining the gas composition under the needed pressure. While the preference is that the material be impermeable, as the surrounding atmosphere with regard to the gas container 2 is the same as what is stored in the gas container 2, a certain degree of permeability is acceptable.

The preferred embodiment of the present invention is represented by gas containers 2 that contain a per unit dose of gas composition 4 that can be used in a procedure involving a human or an animal. For purposes of the present invention, the phrase “per unit dose” refers to the standard amount of gas composition 4 that will be utilized during one test, procedure or surgery. More specifically, the amount of gas composition 4 in the internal reservoir 3 of the gas container 2 is sufficient to dispense an individual unit dose of the gas composition 4 thereby making the gas container a one time use gas container.

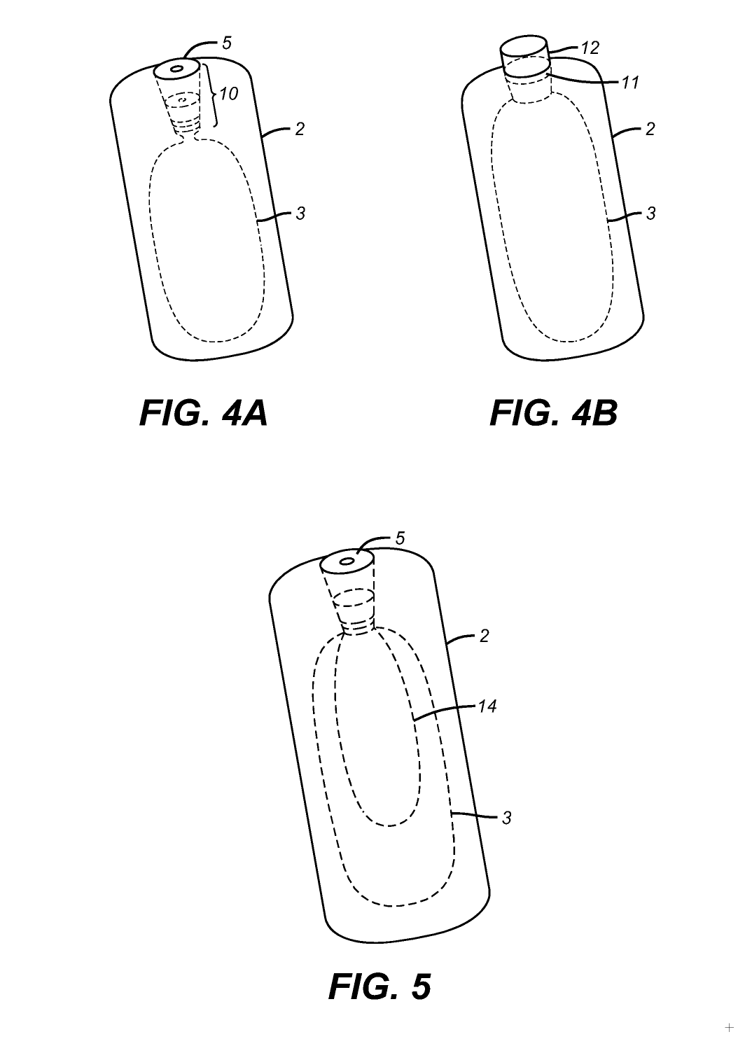

With regard to the means for allowing the withdrawal of the gas composition 5 from the gas container 2, the means 5 may comprise a valve 10 (as shown in FIG. 4a) or a barrier that is capable of being punctured 11 to allow for the withdrawal of the gas composition 4 (as shown in FIG. 4b). Note that in order to allow for the gas composition to be withdrawn from the internal reservoir 3, the means for allowing the withdrawal of the gas composition 5 must be in fluid communication with the internal reservoir 3 where the gas composition 4 is stored. When the means for allowing the withdrawal of the gas composition 5 comprises a valve 10, the valve 10 utilized can be characterized as an outlet valve 10 of the type that when the valve is axially and elastically urged towards the exterior of the gas container 2, the gas container 2 is closed (the gas composition cannot exit the gas container 2) since the valve 10 is in a sealed or closed position and when the valve 10 is forced axially towards the interior of the gas container 2, the gas container 2 is open thereby allowing for the exit of the gas composition 5 from the internal reservoir of the gas container 2. Such valves 10 are known to those skilled in the art and are commonly of the type referred to as “aerosol valves”.

With regard to the means 5 that comprises a valve 10, the valve 10 may be a manually operated valve having an actuating button (not shown) and having a coupling member 12 for connecting a secondary device (not shown) to the gas container 2, typically through a delivery coupler on the secondary device. The secondary device is secured into place and then the actuating button is depressed to allow for the passage of the gas composition 4 which is under pressure into the secondary device. The normal position of the valve 10 is the closed position (the valve 10 when resting is urged towards the exterior of the gas container 2 so that the gas composition cannot leave the internal reservoir 3 of the gas container 2). In order to open the valve 10 and withdraw the gas composition 4, force must be applied to cause the valve 10 to be urged towards the interior of the gas container 2. Such force results from the pressure of a secondary device upon the valve 10 or the release caused by the actuating button. Such secondary devices are known to those skilled in the art and will typically be secondary devices which are the actual tool which will be utilized to deliver the gas composition to the human or animal in question. More specifically, these secondary devices will typically be syringes or syringes that also include a filter unit, each being capable to forming a tight juncture with the valve 10 (often through a coupling member 12) thereby permitting the filling of the syringe or syringe with filter unit without loss of the gas composition. As used herein, the phrase “tight junction” refers to a junction created between a needle, cannula or formed section or tip of the syringe or filter unit and the valve 10. Typically, secondary devices such as those described above include a delivery coupler for connection to the valve 10 of the gas container 2. The delivery coupler interacts with the valve 10 such as to allow the secondary device and valve 10 to be brought together in a tight junction without actually actuating the valve 10 of the gas container 2 which will remain closed as the secondary device is not urging the valve 10 to the open position. A further action of bringing together in a relative axial manner the valve 10 and gas container 2 results in either a portion of the secondary device (such as a needle, cannula or formed section of the syringe or syringe and filter unit) urging the valve 10 to the open position or from the depression of an actuating button thereby allowing for the gas composition 4 to flow from the internal reservoir 3 of the gas container 2 into the interior of the syringe, through the filter unit first in those embodiments where the secondary device also comprises a filter unit.

In many instances, the valve 10 of the gas container is a female valve provided with a distribution orifice, a peripheral gasket, and an actuating cavity capable of receiving one end of a bifunctional delivery tube for actuating the valve and for ejecting the gas composition (not shown). In the embodiment where the valve is a female valve, the secondary device may also include a connection port having, on one side, a bifunctional delivery tube compatible with the female valve of the gas container and, on the other side, a connecting means compatible with the secondary device.

In a still further embodiment, the secondary device can have a needle for piercing a septum or membrane 11 on an outlet coupling that is provided as a part of the valve 10. Alternatively, the secondary device can have a tapered tip for attaching to an outlet coupling and for receiving a needle or other delivery outlet or device.

The gas composition to be stored in the present device 1 can be any gas composition 4 that requires sterile transport and storage in small amounts. As used herein, the phrase “small amounts” refers to amounts in the range of from about 10 ml to about 100 ml, preferably from about 35 ml to about 45 ml. As the gas composition 4 when added to the internal reservoir 3 of the gas container 2 is substantially free of contaminants, the desire is to maintain this gas composition substantially free of contaminants. As used herein, the terms “contamination” or “contaminants” refers to the inclusion of components into the gas composition 4 which are not an intended part of the gas composition 4. Furthermore, as used herein, the phrase “gas composition” is intended to include not only a pure or substantially pure gas (for example a gas composition that is one hundred percent pure sulfur hexafluoride or pure perfluoropropane) but also to a plurality of components that form the gas composition (two or more components; for example a gas composition that is twenty percent oxygen and eighty percent of sulfur hexafluoride or twenty percent oxygen and eighty percent perfluoropropane). Also, as used herein, the phrase “substantially pure” refers to compositions in which there may be small inconsequential amounts of non-reactive components in the gas composition 4, specifically, gas compositions in which the main component is from about 99.0 to about 99.9 percent of the composition and the remaining non-reactive components make up one or less than one percent of the final composition. Non-limiting examples of the types of gas compositions contemplated to be within the scope of the present invention include nitric oxide, nitric oxide-releasing compounds, carbon dioxide, perfluoropropane, perfluorobutane, perfluoroethane, helium, and sulfur hexafluoride or mixture of one or more of these diluted with air and/or oxygen. For purposes of the present invention, the one or more gases can be diluted to a range where the one or more gases make up from about 5% to about 95% of the final composition with the remaining amount being air and/or oxygen. In one advantageous embodiment, the gas composition is one pure gas.

In another advantageous embodiment, the gas is substantially free of oxygen and air and is at least 70% pure, preferably 90% pure and even more preferably 97% pure by volume.

One goal with regard to the present invention is to provide a gas composition 4 that remains contaminant free for a long period of time—in other words, the increase the shelf life of the gas composition 4. In a preferred embodiment, utilizing the device 1 of the present invention, the gas composition 4 has a shelf life of at least one year, preferably at least one and half years.

As shown in FIG. 1, the gas container 2 is placed in an outer package 6 that is capable of being sealed to create a defined enclosed cavity 7 of sufficient size and shape to fully contain the gas container 2 within the confines of the cavity 7. As noted previously, the sealed cavity 7 will have the gas container 2 disposed therein with the gas container 2 surrounded by an atmosphere that is the same as the gas composition 4 stored in the internal reservoir 3 of the gas container 2. The outer package 6 is fabricated of a material characterized as having a gas transmission rate sufficient to essentially prevent the gas composition 4 from diffusing out of the cavity 7 of the outer package 6 and to essentially prevent atmospheric contaminants from diffusing into the cavity 7 of the outer package 6. The outer package 6 is preferably made from a material that is substantially impermeable to oxygen and other atmospheric gases and substantially impermeable to sterilizing gases such as ethylene oxide. While the outer package 6 may be fabricated out of any material that meets the above criteria, preferably the outer package 6 is fabricated from metal foil, an aluminized foil laminate, or a laminates having one or more metallized layers of nylon, oriented polypropylene (OPP), polyethylene (PE), ethylene vinyl alcohol (EVOH), polyethylene terephthalate (PET), low density polyethylene (LDPE), medium density polyethylene (MDPE), and/or cellophane. In addition, a lacquer coating can also be used to create a cold seal.

Note that the outer package 6 may be in the form of a flexible material package such as a bag that can be sealed or in the form of a less flexible formed package with a top that can be sealed. Preferably the cavity 7 in the outer package 6 will be in a shape that corresponds to the shape of the gas container 2. More specifically, when the gas container 2 is cylindrical in form, the outer package 6 will be rectangular in shape, having a bottom portion, a rectangular cavity that sits down in the rectangular shape and a rectangular planar top portion that allows for the outer package 6 to be completed sealed. Note however, that the present invention is not meant to be limited by the shape of the outer package. For example, the bottom portion of the outer package can conform more specifically to the shape of the gas container 2 (e.g., it can be round when the gas container is round). In addition, the top portion does not have to be planar. For example, the outer package 6 can comprise two equal parts which when placed together form a cavity 7 that fits around the gas container 2.

Once the cavity 7 is created, the gas container 2 which holds the gas composition 4 is placed in the cavity 7 and the cavity 7 is sealed. An additional amount of the gas composition 4 that corresponds in composition to that held in the gas container 2 is then placed in the remaining space of the cavity 7 to displace any contaminants that may be present in the cavity 7 once the outer package 6 is sealed. This is achieved by sealing the package 6, evacuating the contents (air) in the cavity 7 and replacing these contents within the cavity 7 with an amount of the gas composition 4 that is sufficient to fill the cavity. This can be facilitated by including an outer package valve 13 which will allow for removal and input. Regardless of the shape that the parts of the outer package take, the parts may be sealed using an adhesive layer or methods such as heat sealing (depending upon the type of material that the outer package is made of). The outer package 6 is fabricated from a material that will prevent the gas container 2 within the cavity 7 from coming into contact with any contaminants that are external to the outer package 6.

Once the device 1 is assembled, the entire device 1 may be sterilized by any means known in the art taking into consideration the components of the device 1 and the materials from which they are made. The present invention is not meant to be limited by the means for sterilization. In many instances, the sterilization will be carried out using ethylene oxide or UV radiation.

In a second embodiment of the present invention, the above noted elements are the same with the exception that the gas container utilized further comprises a flexible internal pocket 14 which contains the gas composition 4 under pressure, the flexible internal pocket 14 being connected to the outlet valve 5 of the gas container 2 and to the exterior of the pocket (but to the interior of the gas container 2), and an amount of thrust gas (in the gaseous state) capable of pressurizing the gas composition 4 contained in the pocket, the thrust gas being the same as the gas composition and positioned between the flexible internal pocket and the walls of the internal reservoir 3 of the gas container 2. Therefore, the thrust gas does not present any risk of contamination of the gas composition 4.

The pressure of the gas composition 4 in the gas container is slightly greater than atmospheric pressure, so as to prevent any accident or any premature loss during the assembly and the filling of the secondary device. The amount of thrust gas is capable of maintaining in the pocket (when the latter contains the gas composition 4) an excess pressure (with respect to atmospheric pressure) of gas composition of less than 2000 hPa, in particular of the order of 600 to 1800 hPa. For this reason it is not necessary to provide a pressure reducing valve or a manometer, nor a stop valve for the filling of the syringe from the cartridge.

The initial pressure of the thrust gas in the gaseous state is capable of compressing any individual quantity of gas composition 4. In practice, this initial pressure may be adapted so that, at the end of distribution of the individual quantity of treatment fluid, the residual pressure of the thrust gas corresponds to atmospheric pressure or is slightly greater than atmospheric pressure, for example of the order of 1050 hPa. The corresponding slight excess pressure (of the order of 20 to 70 hPa) is found in practice to be generally sufficient to expel all the gas composition contained in the pocket.

Also provided is a method for preparing a gas composition device 1 for the sterile transport and storage of a gas composition 4 to be used with regard to a human or an animal. The method is carried out by first forming an outer package 6 from a substantially gas impermeable material such that the outer package 6 contains a cavity 7. A prefilled gas container 2 that contains at least a unit dose of a gas composition 4 that is substantially free of contaminants is then placed in the cavity 7 of the outer package 6. The gas container 2 has an internal reservoir 3 where the gas composition 4 is held and a means for allowing for the withdrawal of the gas composition 5. Once the gas container 2 having the gas composition 4 disposed therein is placed within the cavity 7 of the outer package 6, the outer package 6 is sealed using any means for sealing known in the art. After the outer package 6 is sealed, the atmosphere within the sealed outer package 6 is evacuated and replaced with the same type of gas composition 4 as stored in the internal reservoir 3 of the gas container 2 thereby filling the cavity 7 of the sealed outer package 6 with the additional gas composition 4.

The device of the present invention can be utilized in a variety of different methods which require the injection or forming of a gas bubble in the body of a patient (a human or an animal). When utilizing one or more of the above noted devices 1, the method comprises the first step of supplying for use the device 1 as described above. Once the device 1 is secured, the gas container 2 is removed from the outer package 6 of the device 1. The gas composition 4 is then withdrawn from the internal reservoir 3 of the gas container 4 utilizing a secondary device as defined hereinbefore having a delivery outlet using the means for withdrawing the gas composition 5. Once the gas composition 4 is placed in the secondary device, the delivery outlet of the secondary device is placed into the patient and the gas composition is injected into the patient via the delivery outlet of the secondary device thereby forming a bubble of the gas composition within the patient.

In addition, the device of the present invention can be utilized in a method for forming a gas bubble in the eye of a patient (human or animal) during an eye surgery such as for pneumatic retinopexy. With regard to this method, the first step of the method includes supplying for use the device 1 as described above. Once the device 1 is secured, the gas container 2 is removed from the outer package 6 of the device 1. The gas composition 2 is then withdrawn from the internal reservoir 3 of the gas container 4 utilizing a secondary device as defined hereinbefore having a delivery outlet using the means for withdrawing the gas composition 5. Once the gas composition 4 is placed in the secondary device, the delivery outlet of the secondary device is placed into the patient's eye and the gas composition 4 is injected into the patient's eye via the delivery outlet of the secondary device thereby forming a bubble of the gas composition within the patient's eye.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

What is claimed is:

1. A gas composition device for the transport and storage of a gas composition to be used with regard to a human or an animal, the device consisting essentially of:
a) a gas composition that is substantially free from contaminants;
b) a gas container having an internal reservoir disposed therein that holds the gas composition and a means for allowing the withdrawal of the gas composition, and
c) an outer package capable of being sealed to create a defined enclosed cavity of sufficient size and shape to fully contain the gas container within the confines of the cavity, the cavity, once created, having disposed therein the gas container which holds the gas composition along with an additional amount of the gas composition contained in the gas container to displace any contaminants that may be present in the cavity once the outer package is sealed, the outer package being fabricated from a material that will prevents the gas container within the cavity from coming into contact with any contaminants that are external to the outer package.

2. The device of claim 1, wherein the outer package is fabricated of a material characterized as having a gas transmission rate sufficient to essentially prevent the gas composition from diffusing out of the cavity of the outer package and to essentially prevent atmospheric contaminants from diffusing into the cavity of the outer package.

3. The device of claim 1, wherein the gas composition is a pure gas.

4. The device of claim 1, wherein the gas composition is selected from nitric oxide, nitric oxide-releasing compounds, carbon dioxide, perfluoropropane, perfluorobutane, perfluoroethane, helium, and sulfur hexafluoride.

5. The device of claim 1, wherein the gas container is a gas cylinder having a first end and a second end with the internal reservoir disposed there between and the means for allowing the withdrawal of the gas composition being disposed at either the first end or the second end of the gas cylinder.

6. The device of claim 5, wherein the gas cylinder is a compressed gas cylinder having a diameter of between ½ and 2 inches.

7. The device of claim 6, wherein the outer package is fabricated from metal foil, an aluminized foil laminate, and a laminate having at least one metallized layer of at least one layer of nylon, polypropylene, ethylene vinyl alcohol, polyethylene terephthalate, low density polyethylene, medium density polyethylene or cellophane.

8. The device of claim 1, wherein the device is sterilized by means of ethylene oxide and UV radiation.

9. The device of claim 1, wherein the means for allowing the withdrawal of the gas composition comprises a valve or a material that is capable of being punctured to allow the withdrawal of the gas composition.

10. The device of claim 9, wherein the gas container has a valve that may be coupled to a secondary device to allow for the supply of the gas composition from the internal reservoir of the gas container to be withdrawn into the secondary device.

11. The device of claim 10, wherein the secondary device is a syringe having an optional sterilization device attached thereto.

12. The device of claim 1, wherein the amount of gas composition in the internal reservoir of the gas container is sufficient to dispense an individual unit dose of the gas composition.

13. The device of claim 1, wherein the gas composition has a purity of at least about 95%.

14. The device of claim 1, wherein the gas composition has a shelf life of at least one year.

15. The device of claim 1, wherein the container is made out of material selected from metal foil, an aluminized foil laminate, and a laminate having at least one metallized layer of at least one layer of nylon, polypropylene, ethylene vinyl alcohol, polyethylene terephthalate, low density polyethylene, medium density polyethylene or cellophane.

16. The device of claim 1, wherein the gas composition is substantially free of oxygen and is at least about 93% by volume pure.

17. The device of claim 1, wherein the gas container further comprises a flexible internal pocket placed within the internal reservoir of the gas container, the flexible internal pocket containing the gas composition of the present device under pressure, the flexible internal pocket being connected to the means for withdrawing the gas composition from the gas container and to the exterior of the flexible internal pocket, and an amount of thrust gas that is capable of pressurizing the gas composition contained in the flexible internal pocket, the thrust gas being positioned between the flexible internal pocket and the walls of the internal reservoir of the gas container and being the same in composition as the gas composition in the flexible internal pocket.

18. A method of forming a gas bubble in the body of a patient, the method comprising the steps of:
A) utilizing a device consisting essentially of:
a) a gas composition that is substantially free from contaminants;
b) a gas container having an internal reservoir disposed therein that holds the gas composition and a means for allowing the withdrawal of the gas composition, and
c) an outer package capable of being sealed to create a defined enclosed cavity of sufficient size and shape to fully contain the gas container within the confines of the cavity, the cavity, once created, having disposed therein the gas container which holds the gas composition along with an additional amount of the gas composition contained in the gas container to displace any contaminants that may be present in the cavity once the outer package is sealed, the outer package being fabricated from a material that will prevents the gas container within the cavity from coming into contact with any contaminants that are external to the outer package.
B) removing the gas container from the outer package,
C) withdrawing the gas composition from the internal reservoir of the gas container utilizing a secondary device having a delivery outlet and the means for withdrawing the gas composition;
D) introducing the delivery outlet of the secondary device into the patient, and
E) injecting the gas composition via the delivery outlet of the secondary device into the patient thereby forming a gas bubble of the gas composition within the patient.

19. The method of claim 18, wherein the secondary device comprises a syringe wherein said delivery outlet is a needle or cannula.

20. The method of claim 18, wherein the gas composition has a purity of at least about 70% by volume.

21. The method of claim 18, wherein the gas composition gas has a purity of from about 93% to about 98% by volume.

22. The method of claim 18, wherein the gas composition is one pure gas.

23. The method of claim 18, wherein the outer package of the device has a gas permeability to provide a shelf life of the gas composition in the gas container of at least one year.

24. The method of claim 18, wherein the gas composition is selected from nitric oxide, nitric oxide-releasing compounds, carbon dioxide, perfluoropropane, perfluoroethane, perfluorobutane, helium and sulfur hexafluoride.

25. The method of claim 18, wherein the outer package is made from a laminate material having at least one metal foil layer and a layer selected from nylon, polypropylene, polyethylene, ethylene vinyl alcohol, polyethylene terephthalate and cellophane.

26. The method of claim 18, wherein the outer package has a sufficiently low gas permeability whereby the purity of the gas composition in the gas container is substantially unchanged after about one year.

27. The method of claim 18, wherein the material used to make the outer package is substantially oxygen impermeable material.

28. The method of claim 18, wherein the gas container further comprises a flexible internal pocket placed within the internal reservoir of the gas container, the flexible internal pocket containing the gas composition of the present device under pressure, the flexible internal pocket being connected to the means for withdrawing the gas composition from the gas container and to the exterior of the flexible internal pocket, and an amount of thrust gas that is capable of pressurizing the gas composition contained in the flexible internal pocket, the thrust gas being positioned between the flexible internal pocket and the walls of the internal reservoir of the gas container and being the same in composition as the gas composition in the flexible internal pocket.

29. A method of forming a gas bubble in the eye of a patient during eye surgery, said method comprising the steps of
A) utilizing a device consisting essentially of:
a) a gas composition that is substantially free from contaminants;
b) a gas container having an internal reservoir disposed therein that holds the gas composition and a means for allowing the withdrawal of the gas composition, and
c) an outer package capable of being sealed to create a defined enclosed cavity of sufficient size and shape to fully contain the gas container within the confines of the cavity, the cavity, once created, having disposed therein the gas container which holds the gas composition along with an additional amount of the gas composition contained in the gas container to displace any contaminants that may be present in the cavity once the outer package is sealed, the outer package being fabricated from a material that will prevents the gas container within the cavity from coming into contact with any contaminants that are external to the outer package.
B) removing the gas container from the outer package,
C) withdrawing the gas composition from the internal reservoir of the gas container utilizing a secondary device having a delivery outlet and the means for withdrawing the gas composition;
D) introducing the delivery outlet of the secondary device into the patient's eye, and
E) injecting the gas composition via the delivery outlet of the secondary device into the patient's eye so as to form a gas bubble within the eye.

30. The method of claim 29, wherein the gas composition is selected from nitric oxide, nitric oxide-releasing compounds, carbon dioxide, perfluoropropane, perfluoroethane, perfluorobutane, helium and sulfur hexafluoride.

31. The method of claim 29, wherein the gas composition has a purity of at least from about 93% to about 98% by volume.

32. The method of claim 29, wherein the gas composition is a one pure gas.

33. The method of claim 29, wherein the gas container further comprises a flexible internal pocket placed within the internal reservoir of the gas container, the flexible internal pocket containing the gas composition of the present device under pressure, the flexible internal pocket being connected to the means for withdrawing the gas composition from the gas container and to the exterior of the flexible internal pocket, and an amount of thrust gas that is capable of pressurizing the gas composition contained in the flexible internal pocket, the thrust gas being positioned between the flexible internal pocket and the walls of the internal reservoir of the gas container and being the same in composition as the gas composition in the flexible internal pocket.

34. A method for preparing a gas composition device for the sterile transport and storage of a gas composition to be used with regard to a human or an animal, the method comprising the steps of:
a) forming an outer package from a substantially gas impermeable material, the outer package having an internal space;
b) providing a prefilled gas container that contains at least a unit dose of a gas composition that is substantially free of contaminants, the gas container having an internal reservoir where the gas composition is held and a means for allowing the withdrawal of the gas composition;
c) placing the gas container having the gas composition disposed therein within the cavity of the outer package;
d) sealing the outer package; and
e) evacuating the sealed outer package and thereafter filling the cavity of the sealed outer package with additional gas composition.

35. The method of claim 34, wherein the outer package is configured and arranged so as to include a valve that allows for the evacuation of contaminants and the filling of the cavity of the outer package with gas composition.

36. The method of claim 35, wherein the gas container further comprises a flexible internal pocket placed within the internal reservoir of the gas container, the flexible internal pocket containing the gas composition of the present device under pressure, the flexible internal pocket being connected to the means for withdrawing the gas composition from the gas container and to the exterior of the flexible internal pocket, and an amount of thrust gas that is capable of pressurizing the gas composition contained in the flexible internal pocket, the thrust gas being positioned between the flexible internal pocket and the walls of the internal reservoir of the gas container and being the same in composition as the gas composition in the flexible internal pocket.
